(19) United States
(12) Patent Application Publication
(10) Pub. No.: US 2014/0107474 A1
(43) Pub. Date: Apr. 17, 2014

(54) MEDICAL MANIPULATOR SYSTEM

(71) Applicant: OLYMPUS CORPORATION, Tokyo (JP)

(72) Inventors: Ryohei OGAWA, Tokyo (JP); Kosuke KISHI, Tokyo (JP)

(73) Assignee: OLYMPUS CORPORATION, Tokyo (JP)

(21) Appl. No.: 14/134,340

(22) Filed: Dec. 19, 2013

Related U.S. Application Data
(63) Continuation of application No. PCT/JP2012/066830, filed on Jun. 26, 2012.

(30) Foreign Application Priority Data
Jun. 29, 2011 (JP) 2011-144100

(51) Int. Cl. A61B 19/00; A61B 1/00; A61B 1/313

(52) U.S. Cl. CPC A61B 19/2203; A61B 19/5244; A61B 1/313; A61B 1/00045; A61B 1/00039; A61B 1/00009
USPC 600/424

(57) ABSTRACT

A medical manipulator system includes a master manipulator, a slave manipulator, a first image forming device configured to acquire a first image, a second image forming device configured to form a second image, a display device configured to display the first image and the second image, a selection means configured to select one image from the first image and the second image, a transforming means configured to transform the operating command sent by the master manipulator to a corresponding new command such that the selected image coordinate system coincides with the operational coordinate system being used by the master manipulator, and a driving means configured to cause the slave manipulator to move in accordance with the new command.

This application is a continuation application based on a PCT Patent Application No. PCT/JP2012/066830, filed on Jun. 26, 2012, whose priority is claimed on Japanese Patent Application No. 2011-144100, filed on Jun. 29, 2011. The contents of both the PCT Application and the Japanese Application are incorporated herein by reference.

### BACKGROUND OF THE INVENTION

1. Field of the Invention

The present invention relates to a medical manipulator system.

2. Description of Related Art

Conventionally, a medical manipulator system is known as a surgical support system. The medical manipulator system is provided with a master manipulator which is operated by an operator, and a slave manipulator that performs treatment based on the movements of the master manipulator.

For example, in Published Japanese Translation No. 2009-512514 of the PCT International Publication, a medical robotic system is disclosed that is provided with a master manipulator and a slave manipulator, and with a display screen that displays images of treatment objects and the like.

The medical robotic system described in Published Japanese Translation No. 2009-512514 of the PCT International Publication has two master input devices that are manually operated by an operator, and slave arms of which two are connected to each one of the master input devices. Moreover, in this medical robotic system, it is possible to use a switch to switch between slave arms which move correspondingly to the movements of the master input device.

Furthermore, the medical robotic system described in Published Japanese Translation No. 2009-512514 of the PCT International Publication is provided with a mode that operates as a pointing device for moving a cursor that indicates input positions and coordinates on a display screen as an operating mode for the master manipulator.

### SUMMARY OF THE INVENTION

According to a first aspect of the present invention, a medical manipulator system includes a master manipulator, a slave manipulator, a first image forming device, a second image forming device, a display device, a selection means, a transforming means, and a driving means. The master manipulator is configured to send operating commands. The slave manipulator is configured to support a surgical instrument which performs treatment to a treatment object. The first image forming device is configured to acquire a first image which includes a portion of the slave manipulator and the treatment object. The second image forming device is configured to form a second image which is a different image from the first image and which includes an image which corresponds to a portion of the slave manipulator. The display device is configured to display the first image and the second image. The selection means is configured to select one image from the first image and the second image. The transforming means is configured to transform the operating command sent by the master manipulator to corresponding new command such that the selected image coordinate system coincides with the operational coordinate system being used by the master manipulator. The driving means is configured to cause the slave manipulator to move in accordance with the new command.

According to a second aspect of the present invention, the first image forming device according to the first aspect of the present invention is an endoscope. Moreover, the second image forming device creates a virtual manipulator image in which the slave manipulator is displayed such that it appears to be moving in accordance with the new command, and then creates the second image by synthesizing the virtual manipulator image onto a base image which is formed by creating an image of the treatment object.

According to a third aspect of the present invention, the second image forming device according to the second aspect of the present invention updates the base image based on the operating command from the master manipulator, and alters the point of view.

According to a fourth aspect of the present invention, the medical manipulator system according to the second or the third aspect of the present invention is used together with an examination device that forms images of the treatment object while treatment is being performed on the treatment object. Moreover, the second image forming device uses images formed by the examination device during the treatment task as the base image.

According to a fifth aspect of the present invention, the examination device according to the fourth aspect of the present invention is provided in the slave manipulator.

### BRIEF DESCRIPTION OF THE DRAWINGS

FIG. 1 is an overall view showing a medical manipulator system of a first embodiment of the present invention.

FIG. 2 is a block diagram of the medical manipulator system of the first embodiment of the present invention.

FIG. 3 is a view used to illustrate operations when the medical manipulator system of the first embodiment of the present invention is put to use.

FIG. 4 is a block diagram showing the structure of a portion of the medical manipulator system of a second embodiment of the present invention.

### DETAILED DESCRIPTION OF THE INVENTION

### First Embodiment

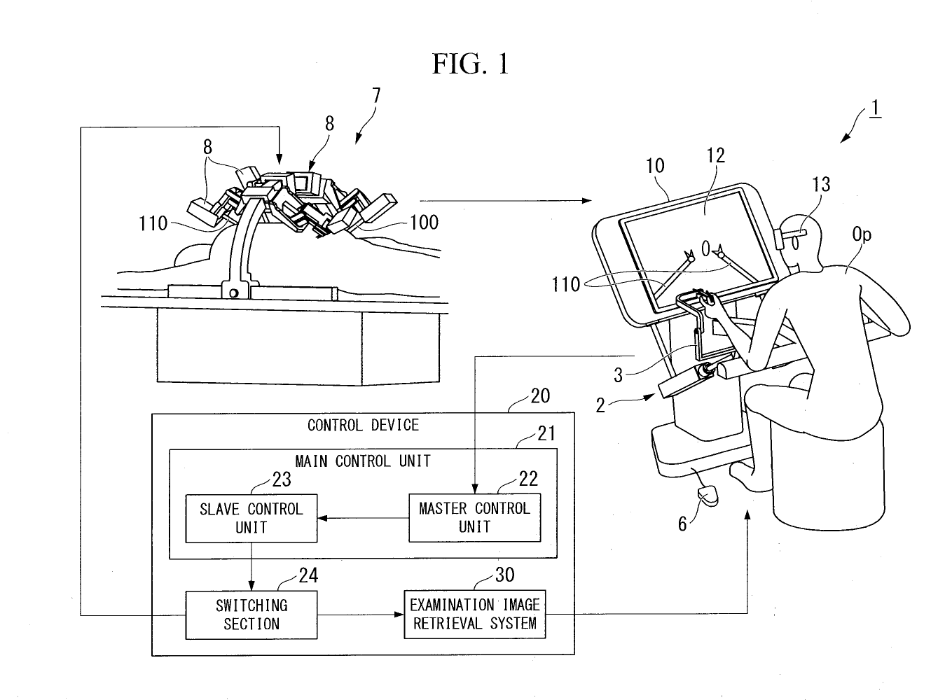

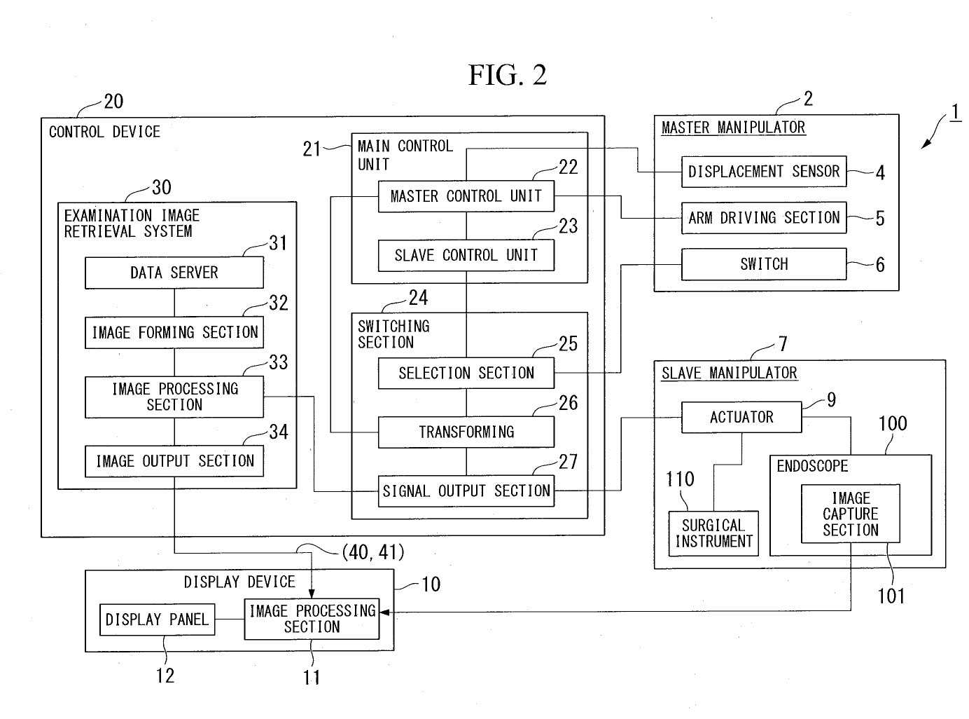

A medical manipulator system 1 of a first embodiment of the present invention will now be described. FIG. 1 is an overall view showing the medical manipulator system of the present embodiment. FIG. 2 is a block diagram of this medical manipulator system.

As shown in FIG. 1 and FIG. 2, the medical manipulator system 1 is formed by a master manipulator 2, a slave manipulator 7, a display device 10, and a control device 20.

The master manipulator 2 is provided in order to transmit movements of an operator Op to the slave manipulator 7. The master manipulator 2 is provided with a master arm 3 that is moved by the operator Op, and with a switch 6 (i.e., selection means) that switches the images displayed on the display device 10.

The master arm 3 has a multi-axial structure that is able to move so as to match the movable range of the slave manipulator 7 and a surgical instrument 110. Furthermore, in the master arm 3, a displacement sensor 4 that emits an electrical signal which shows an amount of change in a position and orientation of the master arm 3, and an arm driving section 5 that operates the master arm 3 in accordance with drive signals from a master control section 22 are mounted (described below).

The electrical signals emitted by the displacement sensor 4 are output to the control unit 20, and form operating commands that are used to operate the slave manipulator 7. In this manner, the master manipulator 2 receives operations made by the operator Op, and sends operating commands based on the operations made by the operator Op.

The switch 6 is configured such that, when it is pressed, the medical manipulator system 1 outputs a predetermined switching signal to the control unit 20. In FIG. 1, the switch 6 is shown on the floor close to the manipulator, however, the input mechanism is not particularly limited, and various types of switch can be selected as is appropriate.

The slave manipulator 7 is provided with a slave arm 8, an endoscope 100 (i.e., a first image forming device), the surgical instrument 110, and an actuator 9 (i.e., a driving means). The endoscope 100 and the surgical instrument 110 are fitted onto the slave arm 8. The actuator 9 operates the endoscope 100, the surgical instrument 110, and the slave arm 8. The actuator 9 that moves the endoscope 100, the surgical instrument 110, and the slave arm 8 operates in accordance with drive signals output from the control unit 20.

The endoscope 100 which is provided in the slave manipulator 7 has an image capture section 101 that acquires images (i.e., first images) that include a portion of the slave manipulator 7 and the treatment object. In the present embodiment, the reference to a ‘portion of the slave manipulator 7’ that is included in the first images indicates a portion of the surgical instrument 110 and a portion of the slave arm 8. Namely, using the image capture section 101, it is possible to acquire images showing the actual process when the treatment object is being treated using the slave manipulator 7. In the present embodiment, the endoscope 100 acquires two images that are capable of forming a stereoscopic image, and outputs these as an electrical signal to the display device 10.

The surgical instrument 110 which is provided in the slave manipulator 7, the surgical instrument 110 is provided in order to perform treatment to the treatment object within the field of view of the image capture section 101. The type of surgical instrument 110 is not particularly limited, and known surgical instruments can be appropriately used in accordance with the type of treatment. The surgical instrument 110 is not limited to be a surgical instrument that is capable of being mounted on the slave manipulator 7. The surgical instrument 110 may be a surgical instrument that cooperates with the slave manipulator 7.

The display device 10 is mounted on the same base as the master manipulator 2, and is located in front of the operator Op. The display device 10 is provided with an image processing section 11, and a display panel 12. The image processing section 11 creates a stereoscopically viewable image based on the two images acquired by the image capture section 101. The display panel 12 is formed, for example, by a liquid crystal panel or an organic EL panel or the like. Namely, the display device 10 is able to display two images that are able to form a stereoscopic image. In the present embodiment, 3-D glasses 13 that separate the two images acquired by the image capture section 101 into an image for each eye are supplied as an accessory to the display device 10. By putting on the 3-D glasses 13 and then looking through the 3-D glasses 13, the operator Op of the medical manipulator system 1 is able to see the image displayed on the display panel 12 as a stereoscopic image. Note that the display panel 12 may also be a panel that provides a stereoscopic view to the naked eye. In the case of the panel that provides a stereoscopic view to the naked eye, there is no need for the 3-D glasses 13 to be supplied as an accessory to the display device 10.

As shown in FIG. 1 and FIG. 2, the control device 20 is provided with a main control section 21, a switching section 24, and an examination image retrieval system 30 (i.e., a second image forming device).

The main control section 21 is electrically connected via a signal wire to the master manipulator 2, the switching section 24, and the examination image retrieval system 30. The main control section 21 is also provided with the master control section 22 and a slave control section 23.

Operating commands sent from the master manipulator 2 are input into the master control section 22. The master control section 22 outputs the input operating commands to the slave control section 23. Furthermore, the master control section 22 changes the orientation of the distal end portion (i.e., the gripping portion) of the master arm 3 by outputting drive signals to the arm driving section 5 in accordance with the orientation of the distal end portion of a virtual manipulator 40 (described below) and the slave manipulator 7 which are displayed on the display device 10.

Operating commands sent from the master control section 22 are input into the slave control section 23. The slave control section 23 also outputs drive signals that cause the slave manipulator 7 to move in accordance with the operating commands. In some cases, the contents (i.e., the coordinate system) of the drive signals output from the slave control section 23 are modified in the switching section 24, and are then output to the slave manipulator 7 (this is described below in detail).

The switching section 24 is provided with a selection section 25, a transforming means 26, and the signal output section 27. The selection section 25 selects either the endoscope device 100 which is provided in the slave manipulator 7, or a virtual endoscope 41 which is provided in the virtual manipulator 40 (described below) as an operating object. The transforming means 26 converts the coordinate system of the drive signals for the operating object selected by the selection section 25. The signal output section 27 outputs to the operating object the drive signals whose coordinate system has been converted by the transforming means 26. Note that the virtual manipulator 40 is a virtual type of manipulator that is created by the examination image retrieval system 30, and is displayed as an image on the display device 10.

The selection section 25 is electrically connected to the switch 6 and, each time the switch 6 is pressed, switches between setting the endoscope device 100 as the operating object and setting the virtual endoscope 41 as the operating object. When the endoscope device 100 is selected as the operating object in the selection section 25, the image acquired by the image capture section 101 of the endoscope device 100 is output to the display device 10. When the virtual endoscope 41 is selected as the operating object in the selection section 25, the image formed by the examination image retrieval system 30 is output to the display device 10.

The transforming means 26 converts the coordinate system of the drive signals such that the coordinate system on the display screen when the selected operating object is being displayed on the display panel 12 of the display device 10 coincides with the operational coordinate system being used by the master manipulator 2. The coordinate system on the display screen is a three-dimensional coordinate system of the stereoscopic image being displayed on the display panel 12. The stereoscopic image at this time appears when it is viewed using the 3-D glasses 13.

The drive signals whose coordinate system is converted by the transforming means 26 form a new command having a different coordinate system from the operating command sent by the master manipulator 2.

When the operating object is the endoscope 100, a drive signal is output from the signal output section 27 to the endoscope 100 and the surgical instrument 110. When the operating object is the virtual endoscope 41, a drive signal is output from the signal output section 27 to the examination image retrieval system 30.

The examination image retrieval system 30 is a system that creates an image from the information obtained when a patient or the like who was the treatment object was examined, and then outputs this to the display device 10 together with an image of the virtual manipulator 40.

As is shown in FIG. 2, the examination image retrieval system 30 is provided with a data server 31, an image forming section 32, an image processing section 33, and an image output section 34. The data server 31 stores information that was obtained when a patient or the like was examined. The image forming section 32 forms a base image which is based on the information stored in the data server 31. The image processing section 33 processes the images formed by the image forming section 32. The image output section 34 outputs to the display device 10 the images processed by the image processing section 33.

In the data server 31, images (i.e., ultrasound images) obtained by ultrasound imaging that was performed prior to an operation on a patient to be treated are stored.

Based on the ultrasound images stored in the data server 31, the image forming section 32 forms a base image that is formed from a three-dimensional image. Drive signals output from the switching section 24 are also input into the image forming section 32.

The image forming section 32 updates the base image in accordance with the drive signals that are output from the switching section 24. For example, when an operator Op uses the master manipulator 2 to move the virtual endoscope 41, or to change the orientation of the virtual endoscope 41, the image forming section 32 sets an image in which the aspect of the virtual endoscope 41 appears to have been altered to a different position and angle as the new base image.

The image processing section 33 selects virtual manipulator images that are able to provide a stereoscopic view that corresponds to the orientation and movement set by the drive signal from the transforming means 26 from a predetermined database. The virtual manipulator image may include an image of a portion of the slave arm 8, an image of a portion of the surgical instrument 110, or an image that imitates the slave arm 8 and the surgical instrument 110. Namely, the virtual manipulator image is an image in which an orientation and movement that correspond to the operations performed by the master manipulator 2 are reflected.

The image processing section 33 superimposes the virtual manipulator image onto the base image so as to create images (i.e., second images) in which the virtual manipulator 40 appears to move on top of the base image based on the operations performed by the master manipulator 2.

The image processing section 33 also outputs the created second images to the image output section 34.

The image output section 34 temporarily stores the second images output from the image processing section 33, and sequentially outputs the temporarily stored second images to the display device 10 at a predetermined frame rate. By doing this, a stereoscopic dynamic image is displayed on the display device 10 in which the virtual manipulator 40 appears to move in accordance with the drive signals (i.e., the new commands) converted by the transforming means 26.

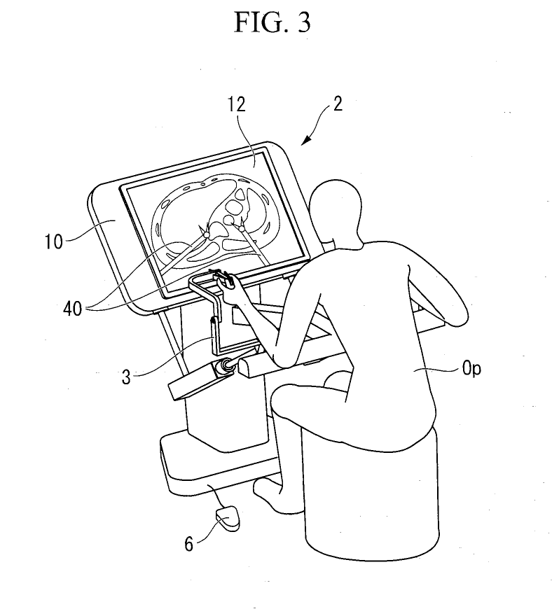

Operations of the medical manipulator system 1 of the present embodiment will now be described. FIG. 3 is a view showing operations when the medical manipulator system 1 is being used.

As shown in FIG. 1, when the medical manipulator system 1 is being used, the operator Op puts on the 3-D glasses 13, and performs treatment to a treatment object while viewing the images displayed on the display panel 12 of the display device 10. By pressing the switch 6, it is possible to switch the image displayed on the display panel 12 between an image (i.e., a first image—see FIG. 1) of the patient being surgically operated on which is acquired by the image capture section 101 (see FIG. 2) of the endoscope 100 which is provided in the slave manipulator 7, and an image (i.e., a second image—see FIG. 3) which is based on an ultrasound image obtained via an examination prior to the surgical operation.

An image of the slave manipulator 7 itself is contained within the first image. An image of the virtual manipulator 40 which is selected by the image processing section 33 is contained within the second image.

When the first image is being displayed, the coordinate system of the slave manipulator 7 on the display device 10 coincides with the operational coordinate system being used by the master manipulator 2. Furthermore, when the second image is being displayed, the coordinate system of the virtual manipulator 40 on the display device 10 coincides with the operational coordinate system being used by the master manipulator 2. Because of this, the operator Op is able to switch operations between the slave manipulator 7 and the virtual manipulator 40 without experiencing any sense of incongruity.

Moreover, by updating the base image in the image forming section 32 in accordance with the movement of the master manipulator 2, in the second image, which is an image that can be viewed stereoscopically, the operator Op is able to recognize an image in which it appears that the operator Op is moving the virtual endoscope 41 to a desired position. In other words, the operator is able to view a pre-surgical operation examination image by performing the same type of operation as the operation that the operator performs with the master manipulator 2 when the endoscope 100 is being used. As a result of this, the operator is able to intuitively contrast the information displayed in the first image with the image which is displayed in the second image, and thereby perform their task efficiently.

### Modified Example 1

Next, a modified example of the above-described first embodiment will be described with reference made to FIG. 1 and FIG. 2.

In this modified example, the structure of the examination image retrieval system 30 is different.

In the data server 31, images (CT images) that are obtained by computerized tomography (CT) using x-rays which is performed on a patient to be treated prior to the surgical operation are stored.

Based on the CT images stored in the data server 31, the image forming section 32 forms a base image which is formed from three-dimensional images.

In this modified example, apart from the fact that the base image is based on CT images, the remaining structure is the same as in the above-described embodiment.

The same type of effects as in the above-described embodiment can also be achieved by means of this structure as well.

### Modified Example 2

Next, another modified example of the above-described first embodiment will be described with reference made to FIG. 1 and FIG. 2.

In this modified example, the structure of the examination image retrieval system 30 is different.

In the data server 31, images (MRI images) that are obtained by nuclear magnetic resonance imaging (MRI) which is performed on a patient to be treated prior to the surgical operation are stored.

The image forming section 32 forms a base image by retrieving a single slice image selected by the operator Op from the MRI images accumulated in the data server 31. One example of a method that may be used to select the base image is a method in which a plurality of slice images that are candidates for the base image are displayed on the display device 10, and an operator then selects one of these slice images by moving the master arm 3.

In this modified example, apart from the fact that the base image is a slice image of an MRI image, the remaining structure is the same as in the above-described embodiment and Modified example 1.

The same type of effects as in the above-described embodiment can also be achieved by means of this structure as well.

Moreover, the MRI image is not limited to slice images (i.e., two-dimensional images), and it is also possible to form a three-dimensional image by employing the same method as in the above-described Modified example 1. The same type of effects as in the above-described embodiment can also be achieved by means of this structure as well.

It is also possible to not only include examination information about the patient in the data server 31 in the examination image retrieval system 30, but to also incorporate three-dimensional (or two-dimensional) CG data such as a general anatomical model of the human body. The same type of effects as in the above-described embodiment can also be achieved by means of this structure as well.

### Second Embodiment

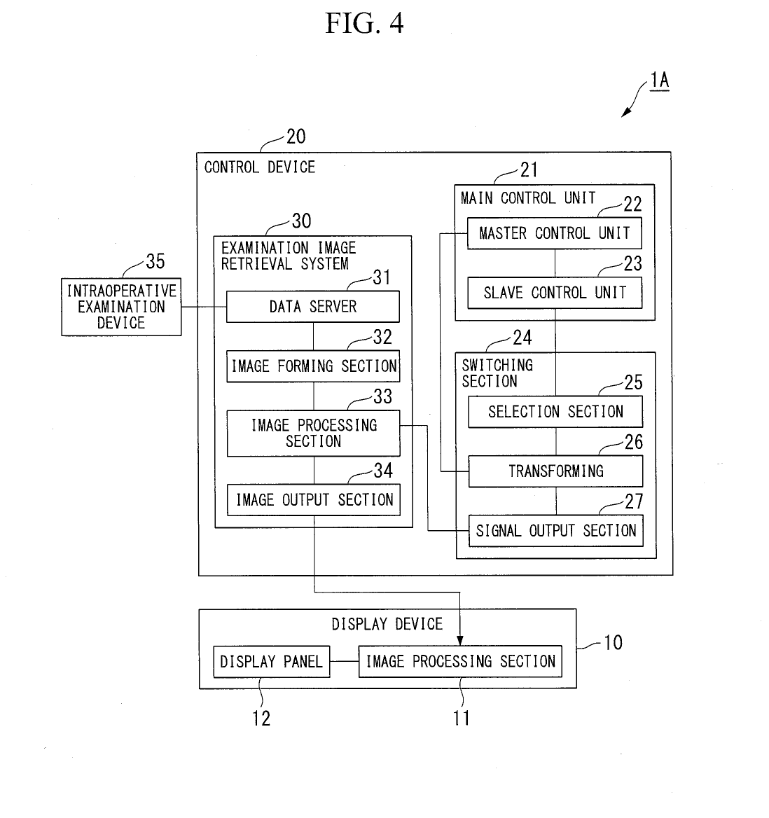

Next, a medical manipulator system 1A according to a second embodiment of the present invention will be described. FIG. 4 is a block diagram showing the structure of a portion of the medical manipulator system of the present embodiment.

As is shown in FIG. 4, the medical manipulator system 1A of the present embodiment differs from that of the first embodiment in that an intraoperative examination device 35 is connected to the examination image retrieval system 30.

The intraoperative examination device 35 is a device such as, for example, an ultrasound endoscope, a fluorescence endoscope, a real-time tomography device, a real-time MRI device and the like. The intraoperative examination device 35 may be mounted on the slave manipulator 7, or may be placed in the vicinity of the patient being treated together with the slave manipulator 7.

The intraoperative examination device 35 makes observations and examinations and the like of a patient during a surgical operation, and stores the information obtained as a result of these observations and examinations in the data server 31. By doing this, the examination image retrieval system 30 forms a base image in the image forming section 32 based on the information acquired by the intraoperative examination device 35. Moreover, in the same way as in the first embodiment, the examination image retrieval system 30 also forms a second image by superimposing an image of the virtual manipulator on top of the base image in the image processing section 33.

According to the medical manipulator 1A of the present embodiment, in the middle of an operation on a patient, information about the patient is acquired by the intraoperative examination device 35, which is a different device from the endoscope 100, and a second image that is formed on the basis of the acquired information is immediately displayed on the display device 10.

Embodiments of the present invention have been described above in detail with reference made to the drawings, however, the specific structure thereof is not limited to these embodiments, and various design modifications and the like may be included insofar as they do not depart from the spirit or scope of the present invention.

For example, the examination device and the type of medical images that are used before and during a surgical operation in the first and second embodiments are not limited to X-ray and MRI devices and images, and the method is not limited provided that a commonly known examination device such as a scintigraphic device, a positron emission tomography (PET) device, a fluoroscopy device, an infrared imaging device, or an angiographic system or the like is used. In these cases as well, the same type of effects as in the above-described embodiment can be achieved.

Moreover, it is also possible for an operating screen that contains buttons or icons which may be used to set the operating mode of the medical manipulator systems 1 or 1A, or for images of documents or diagrams, or for medical information such as an electrocardiogram or the like to be displayed as a second image on the display device 10. In such cases as well, the second image can be operated by an operator operating the master arm 3.

It is also possible for a head-mounted display to be employed as the display device.

Moreover, it is also possible to provide the examination image retrieval system 30 with a gesture input function that, when the master arm 3 makes a specific motion, determines that a specific manipulation is being made.

Moreover, in each of the above-described embodiments, an example is described in which the images displayed on the display panel 12 are switched using the switch 6, however, it is also possible to employ a display device that is provided with a plurality of display panels 12, and for the first image and second image to be displayed on separate display panels 12. In this case, it is possible for the same type of switch 6 as in the above-described first embodiment to be provided, however, instead of the switch 6, it is preferable for a determining means that determines which display panel 12 the operator Op is viewing to be provided in the switching section 24. The determining means preferably has a sensor that detects the direction in which the face or the line of sight of the operator Op is directed towards.

Moreover, in the above-described first embodiment, an example is described in which the arm driving section 5 is provided in order to adjust the orientation of the distal end portion (i.e., the gripping portion) of the master arm 3, however, it is also possible to employ a structure in which an arm driving section is not provided and, instead, the operator is able to manually adjust the orientation of the master arm 3.

Moreover, it is also possible to provide an input device that detects the position and orientation of the master gripping portion using a motion sensor (for example, an optical tracking sensor or the like) without the master arm being provided. In this case as well, it is possible for the operator to manually adjust the orientation of the master gripping portion.

Moreover, it is also possible for the component elements illustrated in each of the above-described embodiments and modified example to be suitably combined into various structures.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A medical manipulator system comprising:
a master manipulator configured to send an operating command;
a slave manipulator supporting a surgical instrument which performs treatment to a treatment object;
a first image forming device configured to acquire a first image which includes a portion of the slave manipulator and the treatment object;
a second image forming device configured to form a second image which is a different image from the first image and which includes an image which corresponds to a portion of the slave manipulator;
a display device configured to display the first image and the second image;
a selection means configured to select one image from the first image and the second image;
a transforming means configured to transform the operating command sent by the master manipulator to a corresponding new command such that the selected image coordinate system coincides with the operational coordinate system being used by the master manipulator; and
a driving means configured to cause the slave manipulator to move in accordance with the new command.

2. The medical manipulator system according to claim 1, wherein
the first image forming device is an endoscope, and
the second image forming device creates a virtual manipulator image in which the slave manipulator is displayed such that it appears to be moving in accordance with the new command, and then creates the second image by synthesizing the virtual manipulator image onto a base image which is formed by creating an image of the treatment object.

3. The medical manipulator system according to claim 2, wherein
the second image forming device updates the base image and alters the point of view based on the operating command from the master manipulator.

4. The medical manipulator system according to claim 2, wherein
the medical manipulator system is used together with an examination device that forms images of the treatment object, while treatment is being performed on the treatment object, and
the second image forming device uses images formed by the examination device during the treatment task as the base image.

5. The medical manipulator system according to claim 4, wherein
the examination device is provided in the slave manipulator.
